(19) United States
(12) Patent Application Publication
(10) Pub. No.: US 2004/0054194 A1
(43) Pub. Date: Mar. 18, 2004

(54) Method for producing 1-pyrrolines

(76) Inventors: Andrew Plant, Berkshire (GB); Johannes Rudolf Jansen, Monheim (DE); Bernd Alig, Konigswinter (DE); Udo Kraatz, Leverkusen (DE); Wolfgang Kramer, Burscheid (DE)

Correspondence Address: BAYER CROPSCIENCE LP, Patent Department, 100 BAYER ROAD, PITTSBURGH, PA, 15205-9741, US

(21) Appl. No.: 10/380,599

(22) PCT Filed: Sep. 10, 2001
(86) PCT No.: PCT/EP01/10423

(51) Int. Cl. C07D 207/04

(52) U.S. Cl. 548/577

(57) ABSTRACT

2,5-Bisaryl-Δ¹-pyrrolines of the formula (1)

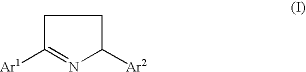

can be prepared by reacting

amides of the formula (II)

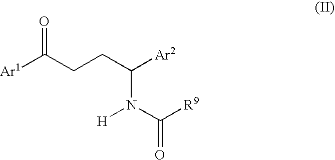

with an N-deacylating agent in the presence of a diluent, where in the formulae

Ar¹, Ar² and R⁹ are as defined in the description.

The present invention relates to a novel process for preparing 2,5-bisaryl-Δ¹-pyrrolines.

Δ1-Pyrrolines, process for their preparation and their use as pesticides have already been described in WO 00/21958, WO 99/59968, WO 99/59967 and WO 98/22438. However, these processes are unsatisfactory with respect to the yields, the practice of the reaction, the number of by-products, the work-up, the amount of waste produced and the energy consumption. Accordingly, there is a constant need for novel processes which overcome one or more of the disadvantages mentioned.

It has now been found that 2,5-bisaryl-Δ¹-pyrrolines of the formula (I)

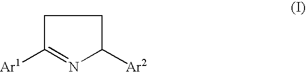

in which

Ar¹ represents the radical

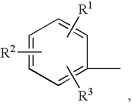

Ar² represents the radical

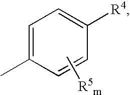

m represents 0, 1, 2, 3 or 4,

R¹ represents halogen, cyano, nitro, alkyl, alkoxy, haloalkyl, haloalkoxy, alkoxyalkyl, —S(O)ₒR⁶ or —NR⁷R⁸,

R² and R³ independently of one another represent hydrogen, halogen, cyano, nitro, alkyl, alkoxy, haloalkyl, haloalkoxy, alkoxyalkyl, —S(O)ₒR⁶ or —NR⁷R⁸,

R⁴ represents halogen or one of the groupings below

(l) —X-A

(m)-B-Z-D

(n) —Y-E,

R⁵ represents halogen, hydroxyl, alkyl, alkoxy, haloalkyl, haloalkoxy, trialkyl-silyl, alkoxycarbonyl, —CONR⁷R⁸, —S(O)ₒR⁶ or —NR⁷R⁸,

X represents a direct bond, oxygen, —S(O)ₒ—, —NR⁶—, carbonyl, carbonyloxy, oxycarbonyl, oxysulphonyl (OSO₂), alkylene, alkenylene, alkynylene, alkylen-oxy, oxyalkylene, oxyalkylenoxy, —S(O)O-alkylene, cyclopropylene or oxiranylene,

A represents phenyl, naphthyl or tetrahydronaphthyl, each of which is optionally mono- or polysubstituted by radicals from the list W¹, or represents 5- to 10-membered saturated or unsaturated heterocyclyl which contains one or more heteroatoms from the group consisting of nitrogen, oxygen and sulphur and is in each case optionally mono- or polysubstituted by radicals from the list W²,

B represents p-phenylene which is optionally mono- or disubstituted by radicals from the list W¹,

Z represents —(CH₂)ₙ—, oxygen or —S(O)ₒ—,

D represents hydrogen, alkyl, alkenyl, alkynyl, haloalkyl, haloalkenyl, haloalkylsulphonyl or dialkylaminosulphonyl,

Y represents a direct bond, oxygen, sulphur, —SO₂—, carbonyl, carbonyloxy, oxy-carbonyl, alkylene, alkenylene, alkynylene, haloalkylene, haloalkenylene, alkylenoxy, oxyalkylene, oxyalkylenoxy or thioalkylene,

E represents hydrogen, alkyl, alkenyl, alkynyl, haloalkyl, haloalkenyl, haloalkylsulphonyl or dialkylaminosulphonyl,

W¹ represents cyano, halogen, formyl, nitro, alkyl, trialkylsilyl, alkoxy, haloalkyl, haloalkenyl, haloalkoxy, haloalkenyloxy, alkylcarbonyl, alkoxycarbonyl, —S(O)ₙR⁶ or —SO₂NR⁷R⁸,

W² represents cyano, halogen, formyl, nitro, alkyl, trialkylsilyl, alkoxy, haloalkyl, haloalkoxy, haloalkenyloxy, alkylcarbonyl, alkoxycarbonyl or —S(O)^{o}R⁶,

n represents 0, 1, 2, 3 or 4,

o represents 0, 1 or 2,

R⁶ represents hydrogen, alkyl or haloalkyl,

R⁷ and R⁸ independently of one another represent hydrogen, alkyl, haloalkyl, or together represent alkylene or alkoxyalkylene,

can be prepared by reacting

amides of the formula (II)

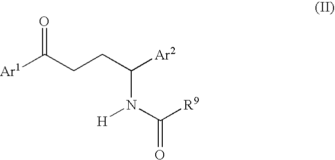

in which

Ar¹ and Ar² are as defined above and

R⁹ represents alkyl, haloalkyl, optionally substituted aryl or aralkyl

with a N-deacylating agent in the presence of a diluent.

It is extremely surprising that 2,5-bisaryl-Δ¹-pyrrolines of the formula (I) can be prepared by the process according to the invention in a smooth reaction in good yields and in high purity.

The process according to the invention has a number of advantages. Thus, the process according to the invention is clearly superior to the processes known from the prior art since it allows a wider range of starting materials to be used (cf. WO 98/22438). Moreover, in the process according to the invention, no regioisomers are formed, giving the products of the formula (I) in a higher yield. A further advantage compared to the prior art is the fact that the process according to the invention dispenses with the use of organometallic compounds, which allows an industrially favourable access to these compounds. Moreover, the use of the process according to the invention offers the advantage that the energy requirements for the practice can be reduced since many reaction steps proceed at from 0° C. to 40° C., frequently even with particular preference at room temperature.

Using N-[1-(4-bromophenyl)-4-(2,6-difluorophenyl)-4-oxobutyl]acetamide as starting material and hydrochloric acid as N-deacylating agent, the course of the process according to the invention can be illustrated by the formula scheme below.

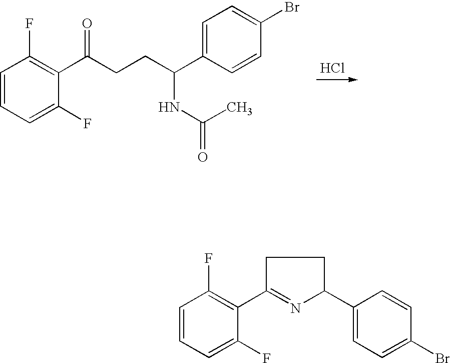

The formula (II) provides a general definition of the amides required as starting materials for carrying out the process according to the invention.

Preferred substituents or ranges of the radicals in the formulae of starting materials of the formula (II) mentioned above and below are illustrated below.

Ar¹ preferably represents the radical

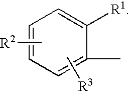

Ar² preferably represents the radical

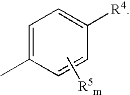

m preferably represents 0, 1, 2 or 3.

R¹ preferably represents halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸.

R² and R³ independently of one another preferably represent hydrogen, halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸.

R⁴ preferably represents fluorine, chlorine, bromine, iodine or one of the groupings below

(I) —X-A

(m)-B-Z-D

(n) —Y-E.

R⁵ preferably represents halogen, hydroxyl, C₁-C₆-alkyl, C₁-C₆-alkoxy, C₁-C₆-haloalkyl, C₁-C₆-haloalkoxy, tri(C₁-C₆-alkyl)silyl, C₁-C₆-alkoxycarbonyl, —CONR⁷R⁸, —S(O)_{O}R⁶ or —NR⁷R⁸.

X preferably represents a direct bond, oxygen, —S(O)ₒ—, —NR⁶—, carbonyl, carbonyloxy, oxycarbonyl, oxysulphonyl (OSO₂), C₁-C₄-alkylene, C₂-C₄-alkenylene, C₂-C₄-alkynylene, C₁-C₄-alkylenoxy, C₁-C₄-oxyalkylene, C₁-C₄-oxyalkylenoxy, —S(O)ₙ—C₁-C₄-alkylene, cyclopropylene or oxiranylene.

A preferably represents phenyl, naphthyl or tetrahydronaphthyl, each of which is optionally mono- to tetrasubstituted by radicals from the list W¹, or represents 5- to 10-membered heterocyclyl which contains 1 or 2 aromatic rings and 1 to 4 heteroatoms, selected from 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyriridinyl, pyridazyl, triazinyl, triazyl, quinolinyl or isoquinolinyl) and is in each case optionally mono- to tetrasubstituted by radicals from the list W².

B preferably represents p-phenylene which is optionally mono- or disubstituted by radicals from the list W¹.

Z preferably represents —(CH₂)ₙ—, oxygen or —S(O)O—.

D preferably represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl) amino sulphonyl.

Y preferably represents a direct bond, oxygen, sulphur, —SO₂—, carbonyl, car-bonyloxy, oxycarbonyl, C₁-C₆-alkylene, C₂-C₆-alkenylene, C₂-C₆-alkynylene, C₁-C₆-haloalkylene, C₂-C₆-haloalkenylene, C₁-C₄-alkylenoxy, C₁-C₄-oxyalkylene, C₁-C₄-oxyalkylenoxy or C₁-C₄-thioalkylene.

E preferably represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl)aminosulphonyl.

W¹ preferably represents cyano, halogen, formyl, nitro, C₁-C₆-alkyl, tri(C₁-C₄-alkyl)silyl, C₁-C₆-alkoxy, C₁-C₆-haloalkyl, C₂-C₆-haloalkenyl, C₁-C₆-haloalkoxy, C₂-C₆-haloalkenyloxy, C₁-C₆-alkylcarbonyl, C₁-C₆-alkoxycarbonyl, —S(O)ₒR⁶ or —SO₂NR⁷R⁸.

W² preferably represents cyano, halogen, formyl, nitro, C₁-C₆-alkyl, tri(C₁-C₄-alkyl)silyl, C₁-C₆-alkoxy, C₁-C₆-haloalkyl, C₁-C₆-haloalkoxy, C₂-C₆-haloalkenyloxy, C₁-C₆-alkylcarbonyl, C₁-C₆-alkoxycarbonyl or —S(O)ₒR⁶.

n preferably represents 0, 1, 2, 3 or 4.

o preferably represents 0, 1 or 2.

R⁶ preferably represents hydrogen, C₁-C₆-alkyl or C₁-C₆-haloalkyl.

R⁷ and R⁸ independently of one another preferably represent hydrogen, C₁-C₆-alkyl, C₁-C₆-haloalkyl, or together represent C₂-C₆-alkylene or C₁-C₄-alkoxy-C₁-C₄-alkylene (for example morpholine),

R⁹ preferably represents C₁-C₄-alkyl, C₁-C₄-haloalkyl, optionally substituted phenyl or aralkyl.

Ar¹ particularly preferably represents the radical

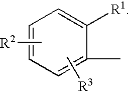

Ar² particularly preferably represents the radical

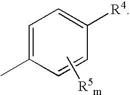

m particularly preferably represents 0, 1 or 2.

R¹ particularly preferably represents fluorine, chlorine, bromine, C₁-C₆-alkyl, C₁-C₆-alkoxy, in each case fluorine- or chlorine-substituted C₁-C₆-alkyl or C₁-C₆-alkoxy.

R² and R³ independently of one another particularly preferably represent hydrogen, fluorine, chlorine, bromine, iodine, C₁-C₆-alkyl, C₁-C₆-alkoxy, in each case fluorine- or chlorine-substituted C₁-C₆-alkyl or C₁-C₆-alkoxy.

R⁴ particularly preferably represents chlorine, bromine, iodine or one of the groupings below

(l) —X-A

(m)-B-Z-D

(n) —Y-E.

R⁵ particularly preferably represents fluorine, chlorine, bromine, iodine, hydroxyl, C₁-C₆-alkyl, C₁-C₆-alkoxy, in each case fluorine- or chlorine-substituted C₁-C₆-alkyl or C₁-C₆-alkoxy, C₁-C₄-alkoxycarbonyl, —CONR⁷R⁸, —S(O)ₒR⁶ or —NR⁷R⁸.

X particularly preferably represents a direct bond, oxygen, sulphur, —SO₂—, carbonyl, carbonyloxy, oxycarbonyl, oxysulphonyl (OSO₂), C₁-C₄-alkylene, C₂-C₄-alkenylene, C₂-C₄-alkynylene, C₁-C₄-alkylenoxy, C₁-C₄-oxyalkylene, C₁-C₄-oxyalkylenoxy, —S(O)ₒ—C₁-C₄-alkylene, cyclopropylene or oxiranylene.

A particularly preferably represents phenyl, naphthyl or tetrahydronaphthyl, each of which is optionally mono- to trisubstituted by radicals from the list W¹, or represents 5- to 10-membered heterocyclyl which contains 1 or 2 aromatic rings and 1 to 4 heteroatoms, selected from 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl) and is in each case optionally mono- to trisubstituted by radicals from the list W².

B particularly preferably represents p-phenylene which is optionally mono- or disubstituted by radicals from the list W¹.

Z particularly preferably represents —(CH₂)ₙ—, oxygen or —S(O)ₒ—.

D particularly preferably represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₄-alkylsulphonyl; or represents di(C₁-C₄-alkyl)aminosulphonyl.

Y particularly preferably represents a direct bond, oxygen, sulphur, —SO₂—, carbonyl, carbonyloxy, oxycarbonyl, C₁-C₆-alkylene, C₂-C₆-alkenylene, C₂-C₆-alkynylene; in each case fluorine- or chlorine-substituted C₁-C₆-alkylene or C₂-C₆-alkenylene; represents C₁-C₄-alkylenoxy, C₁-C₄-oxyalkylene, C₁-C₄-oxyalkylenoxy or C₁-C₄-thioalkylene.

E particularly preferably represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₆-alkylsulphonyl; or represents di(C₁-C₆-alkyl)aminosulphonyl.

W¹ particularly preferably represents cyano, fluorine, chlorine, bromine, iodine, formyl, nitro, C₁-C₄-alkyl, C₁-C₄-alkoxy; in each case fluorine- or chlorine-substituted C₁-C₄-alkyl, C₂-C₄-alkenyl, C₁-C₄-alkoxy or C₂-C₆-alkenyloxy; or represents C₁-C₄-alkylcarbonyl, C₁-C₄-alkoxycarbonyl, —S(O)ₒR⁶ or —SO₂NR⁷R⁸.

W² particularly preferably represents cyano, fluorine, chlorine, bromine, formyl, nitro, C₁-C₄-alkyl, C₁-C₄-alkoxy; in each case fluorine- or chlorine-substituted C₁-C₄-alkyl, C₁-C₄-alkoxy or C₂-C₆-alkenyloxy; or represents C₁-C₄-alkylcarbonyl, C₁-C₄-alkoxycarbonyl, —S(O)ₒR⁶.

n particularly preferably represents 0, 1, 2 or 3.

o particularly preferably represents 0, 1 or 2.

R⁶ particularly preferably represents C₁-C₆-alkyl or in each case fluorine- or chlorine-substituted methyl or ethyl.

R⁷ and R⁸ independently of one another particularly preferably represent C₁-C₆-alkyl, in each case fluorine- or chlorine-substituted C₁-C₆-alkyl, or together represent C₄-C₅-alkylene or represent —(CH₂)₂—O—(CH₂)₂—.

R⁹ particularly preferably represents methyl, ethyl, phenyl or benzyl.

Ar¹ very particularly preferably represents the radical

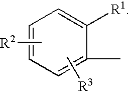

Ar² very particularly preferably represents the radical

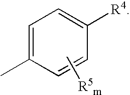

m very particularly preferably represents 0, 1 or 2.

R¹ very particularly preferably represents fluorine, chlorine, bromine, methyl or methoxy.

R² and R³ independently of one another very particularly preferably represent hydrogen, fluorine, chlorine, bromine, methyl or methoxy.

R⁴ very particularly preferably represents chlorine, bromine or one of the groupings below

(l) —X-A

(m)-B-Z-D

(n) —Y-E.

R⁵ very particularly preferably represents fluorine, chlorine, bromine, hydroxyl, methyl, ethyl, methoxy, ethoxy, trifluoromethyl, difluoromethoxy, trifluoromethoxy, —CO₂CH₃ or —SO₂CF₃.

X very particularly preferably represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S(O)ₒ—CH₂— or —S(O)ₒ—(CH₂)₂—.

A very particularly preferably represents phenyl which is optionally mono- or disubstituted by radicals from the list W¹, or represents tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, each of which is optionally mono- or disubstituted by radicals from the list W².

B very particularly preferably represents p-phenylene which is optionally mono-substituted by radicals from the list W¹.

Z very particularly preferably represents oxygen, sulphur or —SO₂—.

D very particularly preferably represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂.

Y very particularly preferably represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S—CH₂— or —S(CH₂)₂—.

E very particularly preferably represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂.

W¹ very particularly preferably represents cyano, fluorine, chlorine, bromine, formyl, methyl, n-butyl, isobutyl, sec-butyl, tert-butyl, methoxy, ethoxy, n-propoxy, n-butoxy, isobutoxy, sec-butoxy, tert-butoxy, trifluoromethoxy, difluoromethoxy, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —OCH₂CF₃, —SCF₃, —SCHF₂, —SOCHF2, —SO₂CHF₂, —SOCF₃, —SO₂CF₃ or —SO₂NMe₂.

W² very particularly preferably represents fluorine, chlorine, bromine, methyl, isopropoxy, tert-butoxy, trifluoromethyl, trifluoromethoxy, difluoromethoxy, trifluoromethylthio, —CO₂CH₃ or —SO₂CF₃.

o very particularly preferably represents 0, 1 or 2,

R⁹ very particularly preferably represents methyl, phenyl or benzyl.

Particularly preferred starting materials for the process according to the invention are the compounds of the formulae

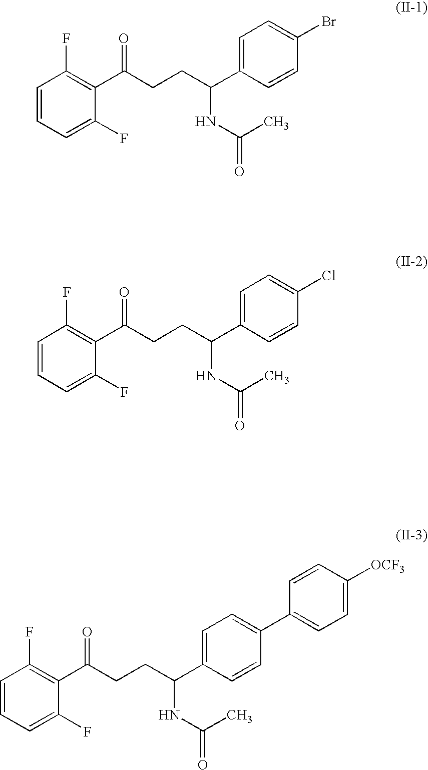

In the definitions mentioned above, oxyalkylene and thioalkylene represent —O-alkyl- and —S-alkyl-, respectively, where the attachment, for example to Ar², is via the oxygen and sulphur atom, respectively, and further substituents may be attached to the alkyl radical, such as, for example, A in —X-A. Alkylenoxy and alkylenethio represent -alkyl-O— and -alkyl-S—, respectively, where the attachment, for example to Ar², is in each case via the alkyl radical and, if appropriate, further substituents may be attached to the oxygen and sulphur atom, respectively, such as, for example, A in —X-A. Oxyalkylenoxy represents —O-alkyl-O.

In the present description, heterocyclyl represents a cyclic hydrocarbon in which one or more carbons are replaced by one or more heteroatoms. Preferred heteroatoms are O, S, N, P, in particular O, S and N.

Preferred, particularly preferred and very particularly preferred are compounds carrying the substituents mentioned under preferred, particularly preferred and very particularly preferred, respectively.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals may be mono- or polysubstituted, where in the case of polysubstitutions the substituents may be identical or different. A plurality of radicals having the same indices, such as, for example, m radicals R⁵ for m>1, can be identical or different.

Halogen-substituted, radicals, such as, for example, haloalkyl, are mono- or polyhalogenated. In the case of polyhalogenation, the halogen atoms can be identical or different. Here, halogen represents fluorine, chlorine, bromine or iodine, in particular fluorine or chlorine.

However, the abovementioned general or preferred radical definitions or illustrations can also be combined with one another as desired, i.e. between the respective ranges and preferred ranges. The definitions apply both to the end products and, correspondingly, to precursors and intermediates.

Some of the amides of the formula (I) required as starting materials for carrying out the process according to the invention are known. Amides of the formula (II-a)

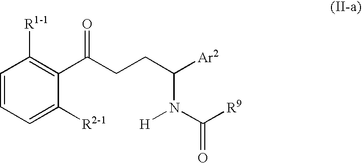

in which

R¹⁻¹ represents fluorine or chlorine,

R²⁻¹ represents hydrogen, fluorine or chlorine and

Ar² and R⁹ are as defined above are novel.

Amides of the formula (II-a) can be prepared by reacting

a) cyclopropanes of the formula (III-a)

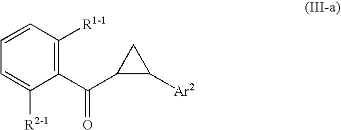

in which R¹⁻¹, R²⁻¹ and Ar² are as defined above, with nitrites of the formula (IV)

R⁹—CN (IV)

in which R⁹ is as defined above

and a protic acid or trimethylsilyl tetrafluoroborate.

Amides of the formula (II) can be prepared analogously. To this end, cyclopropanes of the formula (III)

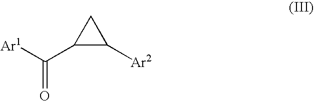

in which Ar¹ and Ar² are as defined above,

are reacted according to process (a).

The formula (III-a) provides a general definition of the cyclopropanes required as starting materials for carrying out the process (a) according to the invention. In this formula, Ar² preferably, particularly preferably and very particularly preferably has those meanings which have already been mentioned in connection with the description of the starting materials of the formula (II) as being preferred, particularly preferred and very particularly preferred, respectively, for these radicals. R¹⁻¹ preferably represents fluorine or chlorine, R²⁻¹ preferably represents hydrogen, fluorine or chlorine.

The formula (IV) provides a general definition of the nitrites required as starting materials for carrying out the process (a) according to the invention. In this formula, R⁹ preferably, particularly preferably and very particularly preferably has those meanings which have already been mentioned in connection with the description of the starting materials of the formula (II) as being preferred, particularly preferred and very particularly preferred, respectively, for these radicals. Especially preferably, R⁹ represents methyl.

Nitriles of the formula (IV) are known.

Protic acids suitable for carrying out the process (a) according to the invention are all acids which are usually used for this purpose. Preference is given to using sulphuric acid.

A trimethylsilyl tetrafluoroborate suitable for carrying out the process (a) according to the invention is the compound of the formula (V)

Me₃Si—N═C⁺—CH₃BF₄⁻ (V).

The reagent of the formula (V) is known (cf. Tetrahedron Lett. 1984, 25, 577-578).

The reaction temperatures for carrying out the process (a) according to the invention can be varied within a relatively wide range. In general, the process is carried out at temperatures between −20° C. and +60° C., preferably between −10° C. and 30° C.

Some of the cyclopropanes of the formula (III) required as starting materials for carrying out the process (a) according to the invention are known. Cyclopropanes of the formula (III-a)

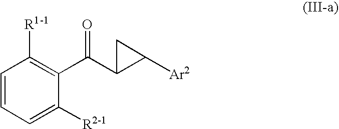

in which R¹⁻¹, R²⁻¹ and Ar² are as defined above

are novel.

Cyclopropanes of the formula (III-a) can be prepared by reacting

b) chalcones of the formula (VI)

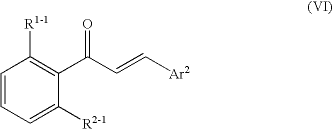

in which R¹⁻¹, R²⁻¹ and Ar² are as defined above

with a trialkylsulphoxonium ylide in the presence of a base and, if appropriate, in the presence of a diluent.

Cyclopropanes of the formula (111) can be prepared analogously.

The formula (VI) provides a general definition of the chalcones required as starting materials for carrying out the process (b) according to the invention. In this formula, Ar² preferably, particularly preferably and very particularly preferably has those meanings which have already been mentioned in connection with the description of the starting materials of the formula (II) as being preferred, particularly preferred and very particularly preferred, respectively, for these radicals. R¹⁻¹ preferably represents fluorine or chlorine, R²⁻¹ preferably represents hydrogen, fluorine or chlorine.

A trialkylsulphoxonium ylide preferably used for carrying out the process (b) according to the invention is trimethylsulphoxonium ylide.

Suitable bases for carrying out the process (b) according to the invention are alkali metal hydrides, alkoxides and hydroxides. Preference is given to using sodium hydride, potassium 2-methyl-2-propoxide, sodium methoxide, or potassium hydroxide, particularly preferably sodium hydride.

Diluents suitable for the process (b) according to the invention are dimethyl sulphoxide, tetrahydrofuran, acetonitrile, toluene or diethylene glycol, and mixtures thereof. Preference is given to using dimethyl sulphoxide (cf. Tetrahedron Asymmetry 1998, 9, 1035).

The reaction temperatures for carrying out process (b) according to the invention can be varied within a relatively wide range. In general, the process is carried out at tempertures between −20° C. and +120° C., preferably between 0° C. and 60° C., particularly preferably between 20° C. and 40° C.

The chalcones of the formula (VI) required as starting materials for carrying out the process (b) according to the invention are known.

When carrying out the process according to the invention, protic acids (cf. J. Org. Chem. 1978, 43, 4593), inorganic bases (cf. J. Chem. Soc. 1964, 4142), hydrazines (cf. J. Org. Chem. 1978, 43, 3711) or biotransformations with enzymes (cf. Appl. Microbiol. Biotechnol. 1997, 47, 650) are used for N-deacylating the amides of the formula (II) during the conversion into pyrrolines of the formula (I). Other customary processes for deacylating amides are described in T. W. Greene, P. G. M. Wuts, Protective Groups in Organic Synthesis (Ed. 3, New York, Wiley 1999, p. 553-555).

Preferred N-deacylating agents are protic acids or organic acids, particularly preferably aqueous hydrochloric acid, aqueous hydrobromic acid or trifluoroacetic acid, very particularly preferably aqueous hydrochloric acid: and, preferably, inorganic bases, particularly preferably barium hydroxide [Ba(OH)₂] and sodium hydroxide (NaOH), and, preferably, biotransformations, particularly preferably using acylases.

The N-deacylation by biotransformations gives the compounds of the formula (I) with an excess of one of the two enantiomers.

Diluents suitable for carrying out the process according to the invention are water or alcohols and mixtures of these. Preference is given to using water, methanol or ethanol, or mixtures of two or three of these three diluents.

When carrying out the process according to the invention, the reaction temperatures can be varied within a relatively wide range. In general, the process is carried out at temperatures between 20° C. and 200° C., preferably between 60° C. and 140° C., particularly preferably between 80° C. and 120° C. If the N-deacylation is carried out enzymatically using acylases, the process is generally carried out between 20° C. and 60° C., preferably between 20° C. and 40° C.

The process according to the invention and the processes (a) and (b) are generally each carried out under atmospheric pressure. However, in each case it is also possible to operate under elevated or reduced pressure.

When carrying out the process according to the invention, in general 2 parts by volume of a protic acid are used per part by volume of a 10% strength (w/v) alcoholic solution of the amide of the formula (II). However, it is also possible to select other ratios of the reaction components. Work-up is carried out by customary methods. In general, the reaction mixture is neutralized with aqueous sodium hydroxide solution and then extracted with ethyl acetate, and the organic phase is dried, filtered and concentrated.

Some of the 2,5-bisaryl-Δ¹-pyrrolines of the formula (I) which can be prepared by the process according to the invention are known. Also known is their use for controlling pests. They are particularly suitable for controlling insects, arachnids and nematodes encountered in agriculture, in forests, in the protection of stored products and the protection of materials and in the hygiene sector (see WO 00/21958, WO 99/59968, WO 99/59967 and WO 98/22438).

2,5-Bisaryl-Δ¹-pyrrolines of the formula (I-a)

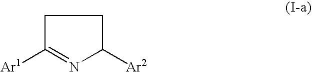

in which

Ar¹ is as defined above,

Ar² represents the radical

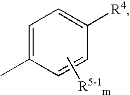

R⁴ and m are as defined above,

R⁵⁻¹ represents hydroxyl, trialkylsilyl, alkoxycarbonyl, —CONR⁷R⁸ or —NR⁷R⁸ and

R⁷ and R⁸ are as defined above

are novel.

Compounds of the formula (I-a) have very good insecticidal properties and can be used both in crop protection and in the protection of materials for controlling unwanted pests, such as insects. They are particularly suitable for controlling insects, arachnids and nematodes encountered in agriculture, in forests, in the protection of stored products and the protection of materials and in the hygiene sector (see WO 00/21958, WO 99/59968, WO 99/59967 and WO 98/22438).

The practice of the process according to the invention is illustrated by the examples below.

### PREPARATION EXAMPLES

### Example 1

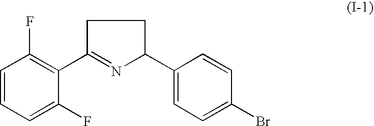

Step 1

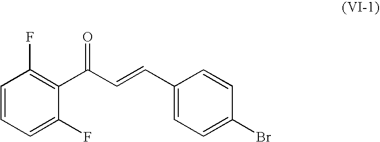

23.40 g of 2,6-difluoroacetophenone (0.15 mol), 27.75 g of 4-bromobenzaldehyde (0.15 mol), 60 ml of methanol and 150 ml of water are initially charged in a three-necked flask. At room temperature, 45 ml of aqueous sodium hydroxide solution (10% strength solution in water) are added dropwise, and the mixture is then stirred at room temperature overnight. The reaction mixture is cooled to 5° C. and the precipitate is filtered off and washed with 100 ml of cold methanol/water (1:3).

This gives 44.77 g (92% of theory) of (2E)-3-(4-bromophenyl)-1-(2,6-difluorophenyl)-2-propen-1-one of melting point 71° C.

HPLC: log P (pH 2.3)=3.98 (98% pure).

¹H-NMR spectrum (D₆-DMSO): δ=7.25-7.35 (3H, m), 7.54 (1H, d), 7.62-7.72 (3H, m), 7.76 (2H, d) ppm.

Step 2

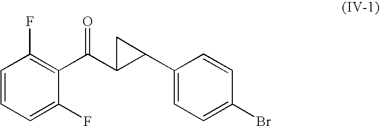

Under an atmosphere of argon, 4.50 g of sodium hydride (80% strength suspension in oil, 0.15 mol) are initially charged in 150 ml of DMSO. 33.0 g of trimethylsulphoxonium iodide (0.15 mol) are added a little at a time. After 2 hours of stirring at room temperature, a solution of 44.40 g of the compound (VI-1) (0.137 mol) in 200 ml of DMSO is added dropwise, and the mixture is stirred further at room temperature overnight. The reaction mixture is stirred into 2 litres of water and extracted twice with in each case 400 ml of ethyl acetate. The combined organic phases are washed once with 200 ml of water, dried over sodium sulphate, filtered and concentrated under reduced pressure. The residue is triturated with isopropanol and filtered off with suction.

This gives 32.36 g (64% of theory) of [2-(4-bromophenyl)cyclopropyl](2,6-difluoro-phenyl)methanone of melting point 64 to 65° C.

HPLC: log P (pH 2.3)=4.24 (97% pure).

¹H-NMR spectrum (CDCl₃): δ=1.57 (1H, m), 1.97 (1H, m), 2.59 (1H, m), 2.77 (1H, m), 6.95 (2H, m), 7.06 (2H, d), 7.40 (3H, m) ppm.

Step 3

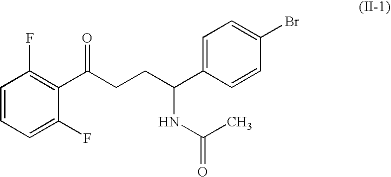

At 0° C., 100 ml of acetonitrile are added dropwise to 39.2 g of concentrated sulphuric acid (98% strength). The mixture is stirred for 60 min, and 33.7 g of the compound (IV-1) (0.1 mol) in 380 ml of acetonitrile are then added dropwise at −10° C. The reaction mixture is allowed to warm to room temperature and stirred at this temperature for another 12 hours. The reaction mixture is poured onto ice/ammonium hydroxide solution (25% strength), and the precipitate is filtered off with suction and dried in the air.

This gives 24.35 g (56% of theory) of N-[1-(4-bromophenyl)-4-(2,6-difluorophenyl)-4-oxobutyl]acetamide as a solid substance of melting point 162-163° C.

HPLC: log P (pH 2.3)=2.67 (91.8% pure).

¹H-NMR spectrum (CD₃CN): δ=1.86 (3H, s), 2.05 (2H, m), 2.91 (2H, m), 4.84 (1H, m), 6.78 (1H, br), 7.04 (2H, m), 7.23 (2H, d), 7.49 (3H, m) ppm.

Step 4

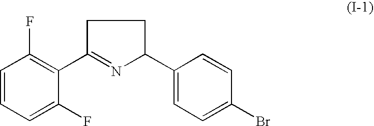

0.57 g (1.4 mmol) of the compound (11-1) is suspended in 5 ml of ethanol. 10 ml of 6N hydrochloric acid is added, and the reaction mixture is heated at 100° C. (oil bath temperature) for 40 hours. After cooling, the reaction mixture is neutralized using aqueous sodium hydroxide solution and then extracted with ethyl acetate. The organic phase is dried over sodium sulphate, filtered and concentrated under reduced pressure.

This gives 0.30 g (64% of theory) of 2-(4-bromophenyl)-5-(2,6-difluorophenyl)-3,4-dihydro-2H-pyrrole.

HPLC: log P (pH 2.3)=2.74 (90% pure).

LC/MS: 336/338

### Example 2

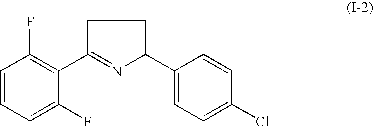

Step 1

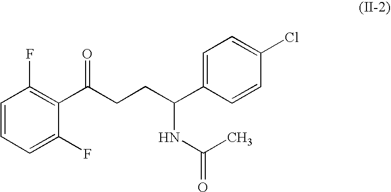

At 0° C., 25 ml of acetonitrile are added dropwise to 9.81 g of concentrated sulphuric acid. The mixture is stirred for 30 min, and 7.32 g of [2-(4-chlorophenyl)-cyclopropyl](2,6-difluorophenyl)methanone (IV-2) (25 mmol) in 90 ml of acetonitrile are then added dropwise at −10° C. The reaction mixture is allowed to warm to room temperature and stirred at this temperature for another 16 hours. The reaction mixture is poured onto ice/45% strength aqueous sodium hydroxide solution, and the precipitate is filtered off with suction.

This gives 4.77 g (50% of theory) of N-[1-(4-chlorophenyl)-4-(2,6-difluorophenyl)-4-oxobutyl]acetamide as a solid.

HPLC: log P (pH 2.3)=2.59 (91.8% pure).

¹H-NMR spectrum (CD₃CN): δ=1.86 (3H, s), 2.08 (2H, m), 2.91 (2H, m), 4.86 (1H, m), 6.80 (1H, br), 7.04 (2H, m), 7.30 (2H, d), 7.32 (2H, d), 7.48 (3H, m) ppm.

The filtrate is extracted twice with in each case 250 ml of ethyl acetate. The combined organic phases are dried over sodium sulphate, filtered and concentrated under reduced pressure. This second batch of product is triturated with n-hexane/ethyl acetate (3:1), and the precipitate is filtered off with suction.

This gives a further 2.14 g (18% of theory) of N-[1-(4-chlorophenyl)-4-(2,6-difluorophenyl)-4-oxobutyl]acetamide as a solid.

HPLC: log P (pH 2.3) 2.59 (68.7% pure)

Step 2

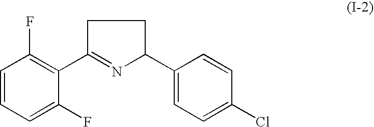

4.50 g (12.8 mmol) of the compound (11-2) are suspended in 50 ml of ethanol. 100 ml of 6N hydrochloric acid are added, and the reaction mixture is heated at 100° C. (oil bath temperature) for 40 hours. After cooling, the reaction mixture is adjusted to pH 11 using ice-cold aqueous sodium hydroxide solution and then extracted with ethyl acetate (2×300 ml). The combined organic phases are dried over sodium sulphate, filtered and concentrated under reduced pressure.

This gives 3.35 g (90% of theory) of 2-(4-chlorophenyl)-5-(2,6-difluorophenyl)-3,4-dihydro-2H-pyrrole.

HPLC: log P (pH 2.3)=2.59 (96.0% pure)

¹H-NMR spectrum (CD₃CN): 1.77 (1H, m), 2.60 (1H, m), 3.03 (2H, m), 5.27 (1H, m), 7.07 (2H, m), 7.33 (2H, d), 7.36 (2H, d), 7.46 (1H, m) ppm.

Compounds of the formulae (I), (II), (III) and (VI) are prepared analogously to the procedures above:

Pyrrolines of the Formula (I)

| | |
|---|---|
| | (I) |
| 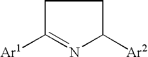 | |

| Ex. No. | Ar¹ | Ar² | logP* | M.p. |
|---|---|---|---|---|
| I-1 | 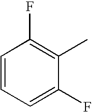 | 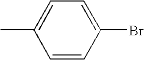 | 2.77 | 48° C. |
| I-2 | 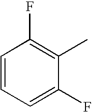 | 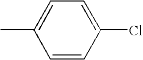 | 2.59 | |
| I-3 | 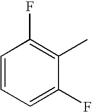 | 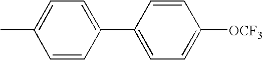 | 4.78 | 112° C. |

Amides of the Formula (II)

| | |
|---|---|
| | (II) |
| 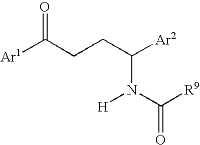 | |

| Ex. No. | Ar¹ | Ar² | R⁹ | logP* | M.p. |
|---|---|---|---|---|---|
| II-1 | 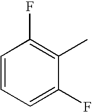 | 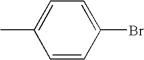 | CH₃ | 2.67 | 162-163° C. |
| II-2 | 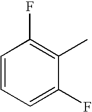 | 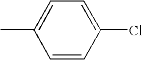 | CH₃ | 2.59 | |
| II-3 | 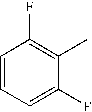 | 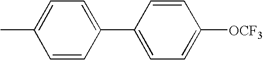 | CH₃ | 3.79 | |

Cyclopropanes of the Formula (III)

| | |
|---|---|
| | (III) |
| 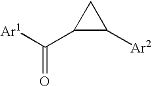 | |

| Ex. No. | Ar¹ | Ar² | logP* | M.p. |
|---|---|---|---|---|
| III-1 | 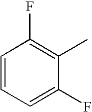 | 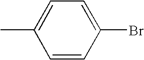 | Br | 4.22 |
| III-2 | 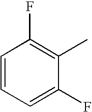 | 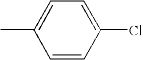 | | |
| III-3 | 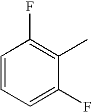 | 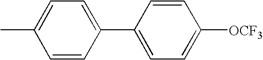 | 5.32 | 72° C. |

Chalcones of the Formula (VI)

| | |
|---|---|
| | (VI) |
| 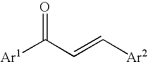 | |

| Ex. No. | Ar¹ | Ar² | logP* | M.p. |
|---|---|---|---|---|
| VI-1 | 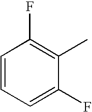 | 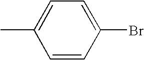 | 3.98 | 71° C. |
| VI-2 | 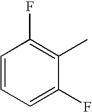 | 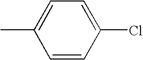 | | |
| VI-3 | 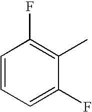 | 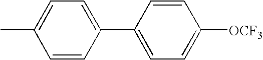 | 4.99 | |

The logp values given in the tables and Preparation Examples above are determined in accordance with EEC Directive 79/831 Annex V .A8 by HPLC (High Performance Liquid Chromatography) on a reversed-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (of 3 to 16 carbon atoms) with known logp values (determination of the logp values by the retention times using linear interpolation between two successive alkanones).

The lambda max values were determined in the maxima of the chromatographic signals using the UV spectra from 200 nm to 400 nm.

1. Process for preparing 2,5-bisaryl-Δ¹-pyrrolines of the formula (I) 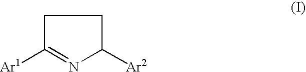
in which
Ar¹ represents the radical 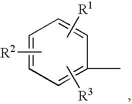
Ar² represents the radical 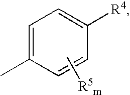
m represents 0, 1, 2, 3 or 4,
R¹ represents halogen, cyano, nitro, alkyl, alkoxy, haloalkyl, haloalkoxy, alkoxyalkyl, —S(O)ₒR⁶ or —NR⁷R⁸,
R² and R³ independently of one another represent hydrogen, halogen, cyano, nitro, alkyl, alkoxy, haloalkyl, haloalkoxy, alkoxyalkyl, —S(O)ₒR⁶ or —NR⁷R⁸,
R⁴ represents halogen or one of the groupings below
(l) —X-A
(m) —B-Z-D
(n) —Y-E,
R⁵ represents halogen, hydroxyl, alkyl, alkoxy, haloalkyl, haloalkoxy, tri-alkylsilyl, alkoxycarbonyl, —CONR⁷R⁸, —S(O)ₒR⁶ or —NR⁷R⁸,
X represents a direct bond, oxygen, —S(O)ₒ—, —NR⁶—, carbonyl, car-bonyloxy, oxycarbonyl, oxysulphonyl (OSO₂), alkylene, alkenylene, alkynylene, alkylenoxy, oxyalkylene, oxyalkylenoxy, —S(O)ₒ-alkylene, cyclopropylene or oxiranylene,
A represents phenyl, naphthyl or tetrahydronaphthyl, each of which is optionally mono- or polysubstituted by radicals from the list W¹, or represents 5- to 10-membered saturated or unsaturated heterocyclyl which contains one or more heteroatoms from the group consisting of nitrogen, oxygen and sulphur and is in each case optionally mono- or polysubstituted by radicals from the list W²,
B represents p-phenylene which is optionally mono- or disubstituted by radicals from the list W¹,
Z represents —(CH₂)ₙ—, oxygen or —S(O)ₒ—,
D represents hydrogen, alkyl, alkenyl, alkynyl, haloalkyl, haloalkenyl, haloalkylsulphonyl or dialkylaminosulphonyl,
Y represents a direct bond, oxygen, sulphur, —SO₂—, carbonyl, carbonyl-oxy, oxycarbonyl, alkylene, alkenylene, alkynylene, haloalkylene, haloalkenylene, alkylenoxy, oxyalkylene, oxyalkylenoxy or thioalkylene,
E represents hydrogen, alkyl, alkenyl, alkynyl, haloalkyl, haloalkenyl, haloalkylsulphonyl or dialkylaminosulphonyl,
W¹ represents cyano, halogen, formyl, nitro, alkyl, trialkylsilyl, alkoxy, haloalkyl, haloalkenyl, haloalkoxy, haloalkenyloxy, alkylcarbonyl, alkoxycarbonyl, —S(O)ₒR⁶ or —SO₂NR⁷R⁸,
W² represents cyano, halogen, formyl, nitro, alkyl, trialkylsilyl, alkoxy, haloalkyl, haloalkoxy, haloalkenyloxy, alkylcarbonyl, alkoxycarbonyl or —S(O)ₒR⁶, n represents 0, 1, 2, 3 or 4,
o represents 0, 1 or 2,
R⁶ represents hydrogen, alkyl or haloalkyl,
R⁷ and R⁸ independently of one another represent hydrogen, alkyl, haloalkyl, or together represent alkylene or alkoxyalkylene,
characterized in that
amides of the formula (II) 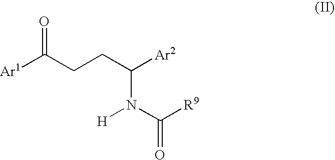
in which
Ar^{1 and Ar}² are as defined above and
R⁹ represents alkyl, haloalkyl, optionally substituted aryl or aralkyl
are reacted with a N-deacylating agent in the presence of a diluent.

2. Process according to claim 1, characterized in that the starting materials used are amides of the formula (II) 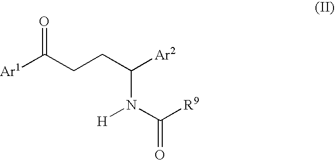
in which
Ar¹ represents the radical 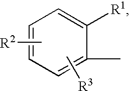
Ar² represents the radical 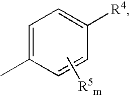
m represents 0, 1, 2 or 3,
R¹ represents halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸,
R² and R³ independently of one another represent hydrogen, halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸,
R⁴ represents fluorine, chlorine, bromine, iodine or one of the groupings below
(l) —X-A
(m)-B-Z-D
(n) —Y-E,
R⁵ represents halogen, hydroxyl, C₁-C₆-alkyl, C₁-C₆-alkoxy, C₁-C₆-haloalkyl, C₁-C₆-haloalkoxy, tri(C₁-C₆-alkyl)silyl, C₁-C₆-alkoxy-carbonyl, —CONR⁷R⁸, —S(O)ₒR⁶ or —NR⁷R⁸,
X represents a direct bond, oxygen, —S(O)ₒ—, —NR⁶—, carbonyl, car-bonyloxy, oxycarbonyl, oxysulphonyl (OSO₂), C₁-C₄-alkylene, C₂-C₄-alkenylene, C₂-C₄-alkynylene, C₁-C₄-alkylenoxy, C₁-C₄-oxyalkylene, C₁-C₄-oxyalkylenoxy, —S(O)ₒ—C₁-C₄-alkylene, cyclopropylene or oxiranylene,
A represents phenyl, naphthyl or tetrahydronaphthyl, each of which is optionally mono- to tetrasubstituted by radicals from the list W¹, or represents 5- to 10-membered heterocyclyl which contains 1 or 2 aromatic rings and 1 to 4 heteroatoms, selected from 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl) and is in each case optionally mono- to tetrasubstituted by radicals from the list W²,
B represents p-phenylene which is optionally mono- or disubstituted by radicals from the list W¹,
Z represents —(CH₂)ₙ—, oxygen or —S(O)ₒ—,
D represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl)aminosulphonyl,
Y represents a direct bond, oxygen, sulphur, —SO₂—, carbonyl, carbonyloxy, oxycarbonyl, C₁-C₆-alkylene, C₂-C₆-alkenylene, C₂-C₆-alkynylene, C₁-C₆-haloalkylene, C₂-C₆-haloalkenylene, C₁-C₄-alkylenoxy, C₁-C₄-oxyalkylene, C₁-C₄-oxyalkylenoxy or C₁-C₄-thioalkylene,
E represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl)aminosulphonyl,
W¹ represents cyano, halogen, formyl, nitro, C₁-C₆-alkyl, tri(C₁-C₄-alkyl)silyl, C₁-C₆-alkoxy, C₁-C₆-haloalkyl, C₂-C₆-haloalkenyl, C₁-C₆-haloalkoxy, C₂-C₆-haloalkenyloxy, C₁-C₆-alkylcarbonyl, C₁-C₆-alkoxycarbonyl, —S(O)ₒR⁶ or —SO₂NR⁷R⁸,
W² represents cyano, halogen, formyl, nitro, C₁-C₆-alkyl, tri(C₁-C₄-alkyl)silyl, C₁-C₆-alkoxy, C₁-C₆-haloalkyl, C₁-C₆-haloalkoxy, C₂-C₆-haloalkenyloxy, C₁-C₆-alkylcarbonyl, C₁-C₆-alkoxycarbonyl or —S(O)ₒR⁶,
n represents 0, 1, 2, 3 or 4,
o represents 0, 1 or 2,
R⁶ represents hydrogen, C₁-C₆-alkyl or C₁-C₆-haloalkyl,
R⁷ and R⁸ independently of one another represent hydrogen, C₁-C₆-alkyl, C₁-C₆-haloalkyl, or together represent C₂-C₆-alkylene or C₁-C₄-alkoxy-C₁-C₄-alkylene (for example morpholine),
R⁹ represents C₁-C₄-alkyl, C₁-C₄-haloalkyl, optionally substituted phenyl or aralkyl.

3. Process according to claim 1, characterized in that the starting materials used are amides of the formula (II) 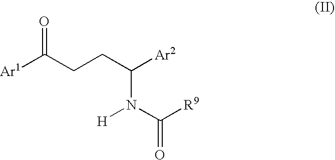
in which
Ar¹ represents the radical 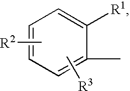
Ar² represents the radical 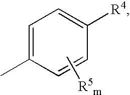
m represents 0, 1 or 2,
R¹ represents fluorine, chlorine, bromine, C₁-C₆-alkyl, C₁-C₆-alkoxy, in each case fluorine- or chlorine-substituted C₁-C₆-alkyl or C₁-C₆-alkoxy,
R² and R³ independently of one another represent hydrogen, fluorine, chlorine, bromine, iodine, C₁-C₆-alkyl, C₁-C₆-alkoxy, in each case fluorine- or chlorine-substituted C₁-C₆-alkyl or C₁-C₆-alkoxy,
R⁴ represents chlorine, bromine, iodine or one of the groupings below
(l) —X-A
(m)-B-Z-D
(n) —Y-E,
R⁵ represents fluorine, chlorine, bromine, iodine, hydroxyl, C₁-C₆-alkyl, C₁-C₆-alkoxy, in each case fluorine- or chlorine-substituted C₁-C₆-alkyl or C₁-C₆-alkoxy, C₁-C₄-alkoxycarbonyl, —CONR⁷R⁸, —S(O)ₒR⁶ or —NR⁷R⁸,
X represents a direct bond, oxygen, sulphur, —SO₂—, carbonyl, car-bonyloxy, oxycarbonyl, oxysulphonyl (OSO₂), C₁-C₄-alkylene, C₂-C₄-alkenylene, C₂-C₄-alkynylene, C₁-C₄-alkylenoxy, C₁-C₄-oxyalkylene, C₁-C₄-oxyalkylenoxy, —S(O)ₒ-C₁-C₄-alkylene, cyclopropylene or oxiranylene,
A represents phenyl, naphthyl or tetrahydronaphthyl, each of which is optionally mono- to trisubstituted by radicals from the list W¹, or represents 5- to 10-membered heterocyclyl which contains 1 or 2 aromatic rings and 1 to 4 heteroatoms, selected from 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl) and is in each case optionally mono- to trisubstituted by radicals from the list W²,
B represents p-phenylene which is optionally mono- or disubstituted by radicals from the list W¹,
Z represents —(CH₂)ₙ—, oxygen or —S(O)ₙ—,
D represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₄-alkylsulphonyl; or represents di(C₁-C₄-alkyl)aminosulphonyl,
Y represents a direct bond, oxygen, sulphur, —SO₂—, carbonyl, car-bonyloxy, oxycarbonyl, C₁-C₆-alkylene, C₂-C₆-alkenylene, C₂-C₆-alkynylene; in each case fluorine- or chlorine-substituted C₁-C₆-alkylene or C₂-C₆-alkenylene; represents C₁-C₄-alkylenoxy, C₁-C₄-oxyalkylene, C₁-C₄-oxyalkylenoxy or C₁-C₄-thioalkylene,
E represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₆-alkylsulphonyl; or represents di(C₁-C₆-alkyl)aminosulphonyl,
W¹ represents cyano, fluorine, chlorine, bromine, iodine, formyl, nitro, C₁-C₄-alkyl, C₁-C₄-alkoxy; in each case fluorine- or chlorine-substituted C₁-C₄-alkyl, C₂-C₄-alkenyl, C₁-C₄-alkoxy or C₂-C₆-alkenyloxy; or represents C₁-C₄-alkylcarbonyl, C₁-C₄-alkoxycarbonyl, —S(O)ₒR⁶ or —SO₂NR⁷R⁸,
W² represents cyano, fluorine, chlorine, bromine, formyl, nitro, C₁-C₄-alkyl, C₁-C₄-alkoxy; in each case fluorine- or chlorine-substituted C₁-C₄-alkyl, C₁-C₄-alkoxy or C₂-C₆-alkenyloxy; or represents C₁-C₄-alkylcarbonyl, C₁-C₄-alkoxycarbonyl, —S(O)ₒR⁶,
n represents 0, 1, 2 or 3,
o represents 0, 1 or 2,
R⁶ represents C₁-C₆-alkyl or in each case fluorine- or chlorine-substituted methyl or ethyl,
R⁷ and R⁸ independently of one another represent C₁-C₆-alkyl, in each case fluorine- or chlorine-substituted C₁-C₆-alkyl, or together represent C₄-C₅-alkylene or represent —(CH₂)₂—O—(CH₂)₂—,
R⁹ represents methyl, ethyl, phenyl or benzyl.

4. Process according to claim 1, characterized in that the starting materials used are amides of the formula (II) 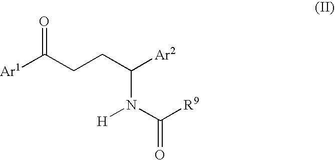
in which
Ar¹ represents the radical 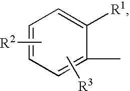
Ar² represents the radical 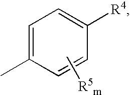
m represents 0, 1 or 2,
R¹ represents fluorine, chlorine, bromine, methyl or methoxy,
R² and R³ independently of one another represents hydrogen, fluorine, chlorine, bromine, methyl or methoxy,
R⁴ represents chlorine, bromine or one of the groupings below
(l) —X-A
(m)-B-Z-D
(n) —Y-E,
R⁵ represents fluorine, chlorine, bromine, hydroxyl, methyl, ethyl, methoxy, ethoxy, trifluoromethyl, difluoromethoxy, trifluoromethoxy, —CO₂CH₃ or —SO₂CF₃,
X represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S(O)ₒ—CH₂— or —S(O)ₒ—(CH₂)₂—,
A represents phenyl which is optionally mono- or disubstituted by radicals from the list W¹, or represents tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, each of which is optionally mono- or disubstituted by radicals from the list W²,
B represents p-phenylene which is optionally mono-substituted by radicals from the list W¹,
Z represents oxygen, sulphur or —SO₂—,
D represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂,
Y represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S—CH₂— or —S(CH₂)₂—,
E represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂,
W¹ represents cyano, fluorine, chlorine, bromine, formyl, methyl, n-butyl, isobutyl, sec-butyl, tert-butyl, methoxy, ethoxy, n-propoxy, n-butoxy, isobutoxy, sec-butoxy, tert-butoxy, trifluoromethoxy, difluoromethoxy, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —OCH₂CF₃, —SCF₃, —SCHF₂, —SOCHF₂, —SO₂CHF₂, —SOCF₃, —SO₂CF₃ or —SO₂NMe₂,
W² represents fluorine, chlorine, bromine, methyl, isopropoxy, tert-butoxy, trifluoromethyl, trifluoromethoxy, difluoromethoxy, trifluoromethylthio, —CO₂CH₃ or —SO₂CF₃,
o represents 0, 1 or 2,
R⁹ represents methyl, phenyl or benzyl.

5. Process according to claim 1, characterized in that the starting material used is the compound of the formula (II-1)

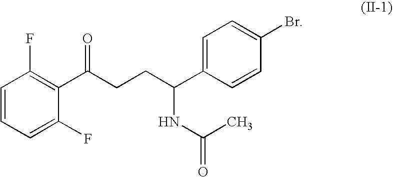

6. Process according to claim 1, characterized in that the starting material used is the compound of the formula (II-2)

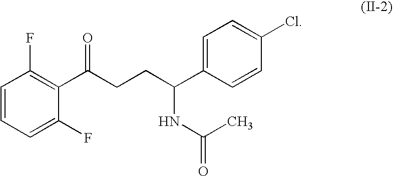

7. Process according to claim 1, characterized in that the starting material used is the compound of the formula (II-3)

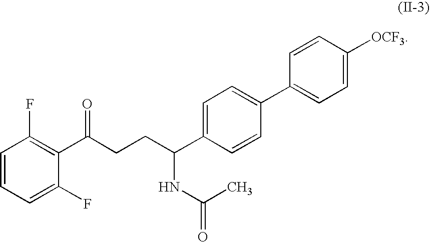

8. Process according to claim 1, characterized in that the N-deacylating agent used is a protic acid, an organic acid, an inorganic base or a biotransformation.

9. Process according to claim 8, characterized in that the N-deacylating agent used is aqueous hydrochloric acid, aqueous hydrobromic acid, trifluoroacetic acid, barium hydroxide, sodium hydroxide or a biotransformation which employs acylases.

10. Process according to claim 9, characterized in that the N-deacylating agent used is aqueous hydrochloric acid.

11. Process according to claim 1, characterized in that the diluent used is water or an alcohol or a mixture of water and alcohols.

12. Process according to claim 11, characterized in that the diluent used is water, methanol, ethanol or a mixture of two or three of these diluents.

13. Process according to claim 1, characterized in that the reaction is carried out at temperatures between 20° C. and 200° C.

14. Compounds of the formula (II-a) 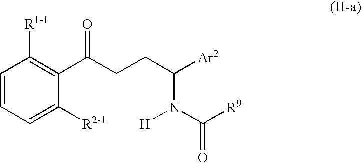
in which
R¹⁻¹ represents fluorine or chlorine,
R²⁻¹ represents hydrogen, chlorine or fluorine and
Ar² and R⁹ are as defined in any of claims 1 to 4.

15. Compounds of the formula (III-a) 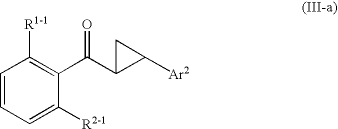
in which
R¹⁻¹ represents fluorine or chlorine,
R²⁻¹ represents hydrogen, chlorine or fluorine and
Ar² is as defined in any of claims 1 to 4.

16. 2,5-Bisaryl-Δ¹-pyrrolines of the formula (I-a) 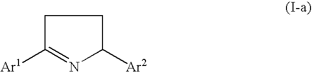
in which
Ar¹ is as defined in any of claims 1 to 4,
Ar² represents the radical 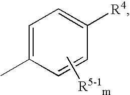
R⁴ and m are as defined in any of claims 1 to 4,
R⁵⁻¹ represents hydroxyl, trialkylsilyl, alkoxycarbonyl, —CONR⁷R⁸ or —NR⁷R⁸ and
R⁷ and R⁸ are as defined in any of claims 1 to 4.
